(19) United States
(12) Patent Application Publication
(10) Pub. No.: US 2009/0318510 A1
(43) Pub. Date: Dec. 24, 2009

(54) TOPICAL DRUG DELIVERY SYSTEM

(75) Inventor: Kevin M. Drizen, Georgetown (CA)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: GLYCOBIOSCIENCES, INC., Georgetown (CA)

(21) Appl. No.: 12/490,841

(22) Filed: Jun. 24, 2009

Related U.S. Application Data
(60) Provisional application No. 61/075,119, filed on Jun. 24, 2008.

(51) Int. Cl. A61K 31/455; A61P 15/00

(52) U.S. Cl. 514/356

(57) ABSTRACT

The present invention relates to compositions for topical delivery of one or more vasoactive vitamins and their methods of preparation and use. More specifically, the composition includes one or more vasoactive vitamins, one or more alkyl hydroxybenzoate preservatives, an alkanol solvent, an alkoxylated alcohol humectant, and a high molecular weight hydrophilic colloid viscosity increasing agent. In various embodiments, the vasoactive vitamins include vitamin B3 compounds.

### CROSS-REFERENCE TO RELATED APPLICATION

This application claims priority to and incorporates by reference the entire contents of U.S. Provisional Application Ser. No. 61/075,119, filed on Jun. 24, 2008.

### FIELD OF THE INVENTION

The present invention relates to topical drug compositions and methods for topical delivery of drugs. Of particular relevance are drug delivery systems for the treatment of sexual dysfunction.

### BACKGROUND

Sexual dysfunction in men and women has been the subject of extensive investigation and review in recent years. The American Foundation for Urologic Disease (AFUD) has developed a classification system to be used by professionals treating female sexual dysfunction. Under this system, female sexual dysfunction includes: hypoactive sexual desire disorder, sexual aversion disorder, sexual arousal disorder, orgasmic disorder and sexual pain disorders. The cause of female sexual dysfunction is not well understood but likely involves vascular, neurological, hormonal and psychogenic factors. Any condition that results in reduced blood flow to the vagina or clitoris can result in sexual dysfunction. Engorgement of these tissues with blood is necessary for producing sexual sensations and for the production of the necessary lubrication.

Neurological disorders can also cause reduced sensation in the vagina and clitoris resulting in sexual dysfunction. As women approach menopause, changes in hormone levels, particularly estrogen and testosterone, can lead to changes in sexual function. Estrogen is needed to help maintain adequate blood flow, muscle tone and lubrication as well as an adequate level of desire. While levels of testosterone are much lower in females than males, adequate levels still are needed in the female to maintain normal desire and libido. Psychological conditions such as anxiety, depression and obsessive compulsive disorder can all be associated with female sexual dysfunction. In addition, some medications used to treat these conditions, such as serotonin re-uptake inhibitors, are associated with reduced desire and libido in women.

In men, sexual dysfunction is usually referred to as erectile dysfunction. Erectile dysfunction is defined as the repeated inability to maintain an erection sufficient for satisfactory intercourse. The are a number of potential causes of erectile dysfunction that involve vascular, neurological, hormonal and psychogenic factors. Vascular function is particularly important since an erection is the result of the penis becoming engorged with blood. In addition to organic causes, erectile dysfunction can be the result of certain medications, in particular certain medications used to treat hypertension.

Treatment options for sexual dysfunction are currently more limited for women than for men. In women, estrogen replacement therapy can relieve many of the symptoms of sexual dysfunction such as vaginal dryness as well as reduce or eliminate symptoms associated with menopause. However, recent studies showing an increase in cardiovascular disease and stroke in women with hormone replacement therapy have raised concerns about the safety of this approach. Treatment with testosterone can increase libido but can also result in weight gain and increased facial hair.

Several orally administered drug products have been developed for treatment of men's erectile dysfunction, such as VIAGRA® (sildenafil citrate), LEVITRA® (vardenafil HCl) and CIALIS® (tadalafil). These phosphodiesterase inhibitors are widely used to treat male sexual dysfunction but trials of these drugs in women with sexual dysfunction have generally had disappointing results.

### SUMMARY OF THE INVENTION

The present invention relates to topical drug compositions containing a suitable vasoactive vitamin, such as a vitamin B3 compound, and methods for effectively delivering said active ingredient to the host, useful for the treatment of sexual dysfunction in both men and women. The invention also relates to methods for formulating or preparing or delivering the composition of the present invention.

In one embodiment of the invention, the composition includes the vitamin B3 compound and an aqueous solution of p-hydroxybenzoic acid methyl ester, ethanol, methoxypolyethylene glycol, hydroxyethylcellulose and carboxymethylcellulose and optionally one or more of hyaluronic acid, propylene glycol, polyvinyl alcohol and polysorbate. In a further embodiment of the invention the aqueous solution includes p-hydroxybenzoic acid methyl ester, ethanol, methoxypolyethylene glycol, hydroxyethylcellulose and carboxymethylcellulose and propylene glycol. In yet a further embodiment of the invention the aqueous solution includes p-hydroxybenzoic acid methyl ester, ethanol, methoxypolyethylene glycol, hyaluronic acid, hydroxyethylcellulose and carboxymethylcellulose and propylene glycol. In still a further embodiment of the invention the composition includes an aqueous solution of p-hydroxybenzoic acid methyl ester, ethanol, methoxypolyethylene glycol, hydroxyethylcellulose and carboxymethylcellulose, polyvinyl alcohol and polysorbate. In yet a further embodiment of the invention the composition includes an aqueous solution of p-hydroxybenzoic acid methyl ester, ethanol, methoxypolyethylene glycol, hyaluronic acid, hydroxyethylcellulose and carboxymethylcellulose, polyvinyl alcohol and polysorbate.

The invention also includes a method for treating sexual dysfunction by topically applying the above described composition to a desired region of the skin of the host. In a particular embodiment, the composition is applied to the vagina and/or clitoral regions of a female desiring treatment for sexual dysfunction prior to sexual activity. In some embodiments, the composition is supplied on the outside of a condom (e.g. in or as a lubricant), where it contacts the genital area of a female desiring treatment for sexual dysfunction during sexual activity.

Another embodiment of the invention is a method for treating erectile dysfunction in males by applying to the skin of the penis the above described composition to deliver the vasoactive vitamin, such as a vitamin B3 compound, directly to the penis. In a preferred embodiment the composition is applied to the penis within about ten minutes prior to sexual activity. In a further embodiment, when the composition is intended for use by males, the number and concentration of viscosity increasing agents and emulsifying agents is increased.

### DETAILED DESCRIPTION OF THE INVENTION

The present invention relates to topical drug compositions and methods for topical drug delivery which promote stability of a drug component and facilitate the penetration of the drug component into the skin of the host. The invention also relates to topical drug compositions containing a suitable vasoactive vitamin, such as a vitamin B3 compound, and methods for effectively delivering said active ingredient to the host. These compositions and methods are useful for the treatment of sexual dysfunction, in both men and women. The invention also relates to methods for formulating, preparing and delivering the composition of the present invention. To minimize irritation, the composition is water based.

The topically applied formulations of the present invention incorporate a drug and deliver pharmacologically effective amounts of the drug to a targeted area of the host or patient. The topical drug composition of the present invention is an aqueous formulation that includes at least one vasoactive vitamin, one or more preservatives, solvents, humectants, viscosity increasing agents, emulsifying agents, and optionally gycosaminoglycans.

Vasoactive vitamins are vitamins that affect either the constriction or relaxation of blood vessels. Suitable vasoactive vitamins include, but are not limited to, vitamin B3 compounds, in particular niacin. In some embodiments of the invention, vitamin B3 compounds are from about 0.05% to about 15% by weight of the composition. In other embodiments, the vitamin B3 component present in the composition is substantially entirely niacin. In further embodiments, the vitamin B3 compounds other than niacin comprise less than about 5% of the total vitamin B3 compounds. Vitamin B3 compounds which are suitable for use as a vitamin B3 compound include the nicotinates. Examples of nicotinates include benzyl nicotinate, inositol hexanicotinate, nicotinic acid, nicotinyl alcohol, xanthine nicotinate, methyl nicotinate, ethyl nicotinate, propyl nicotinate, isopropyl nicotinate, butyl nicotinate, isoamyl nicotinate, hexyl nicotinate, phenyl nicotinate, guaiacyl nicotinate, caffeine nicotinate, xanthinol nicotinate, nicametate citrate, nicotinamide, nicotinuric acid, nicotinyl hydroxamate, tocopheryl nicotinate, and mixtures thereof.

Suitable preservatives include, but are not limited to, alkyl hydroxybenzoates and hydroxyethyl cellulose (HEC). Solvents include but are not limited to alkanols, in particular C₁-C₆ alkanols. Humectants include but are not limited to alkoxylated alcohols. Viscosity increasing agents include but are not limited to hydrophilic colloids, in particular high molecular weight colloids. Emulsifying agents include but are not limited to fatty acid esters.

The invention includes a process of formulating the composition described above. The method of preparation comprises the sequential addition of the following ingredients to water at one or more elevated temperatures in the range of about 55° C. to about 90° C. during stirring said ingredients comprising p-hydroxybenzoic acid methylester, hydroxyethylcellulose, carboxymethylcellulose, methoxypolyethylene glycol and optionally, one or more glycosaminoglycans such as hyaluronic acid; and when the composition is at a temperature below about 60° C. adding a solution of a vitamin B3 compound. In a further embodiment of the process, the process comprises heating water to about 90° C. and adding p-hydroxybenzoic acid methyl ester to the water at the heated temperature. After the addition the water is allowed to cool while stirring at about 450 rpm. When the temperature of the aqueous composition is about 75° C. to about 80° C., hydroxyethylcellulose and carboxymethylcellulose are added and stirring of the aqueous composition at about 450 rpm is continued. When the temperature of the water is about 55° C. to about 60° C. methoxypolyethylene glycol is added and stirring is continued at 450 rpm. A naturally occurring or synthetic vitamin B3 compound is dissolved in any suitable solvent and the resulting solution is added to the aqueous composition with stirring at about 300 rpm. While stirring, propylene glycol is added to the aqueous composition. After addition of the propylene glycol stirring is reduced to a speed of about 90 rpm. In one embodiment, the composition is packaged in a suitable container. In a further embodiment a single dosage amount of the composition is packaged in a disposable container. It is noted that in various embodiments, hyaluronic acid may be added to the aqueous composition.

One of the components of the composition may be an alkyl hydroxybenzoate preservative such as p-hydroxybenzoic acid methyl ester, also known as methyl 4-hydroxybenzoate and as methyl paraben. In the present invention, the free base form of methyl paraben is preferably used. Methyl paraben (free base) is readily available in grades suitable for use in pharmaceutical and cosmetic compositions from a variety of commercial suppliers. In some embodiments, the composition may include hydroxyethyl cellulose as a preservative.

An alkanol solvent, such as ethanol, may be another ingredient in the composition of the invention and may be absolute ethanol (free of water), which is also readily available from commercial sources. Other C₁-C₆ lower alkanols including methanol, butanol, propanol, pentanol and hexanol are also suitable for use in the composition.

Alkoxylated alcohol humectants for use in the invention include methoxypolyethylene glycol, methoxypolypropylene glycol and methoxypolyvinyl glycol. The alkoxylated alcohol preferably has an average molecular weight of from about 300 to about 750 daltons, preferably about 350 daltons. These materials are readily available from commercial sources.

The compositions of the present invention may include one or more glycosaminoglycans. A few examples of suitable glycosaminoglycans include, but are not limited to, hyaluronic acid and chondroitin sulfate.

The composition of the present invention also contains one or more hydrophilic colloids to act as viscosity increasing agents. Suitable hydrophilic colloids are of high molecular weight and include carboxymethylcellulose and hydroxyethylcellulose. Suitable carboxymethylcelluloses that can be used in the invention have a viscosity from about 2,000 cps to about 5,000 cps in a 1% solution. Preferred hydroxyethylcelluloses suitable for use in the invention have a viscosity from about 1,000 cps to about 3,000 cps in a 1% solution.

The hydrophilic colloid component has a high average molecular weight, e.g. on the order of 10,000-15,000 daltons. Suitable materials are available from Hercules Inc. of Wilmington Del. For example the carboxymethylcellulose product AQUALON® CMC 7H3SXF and the hydroxyethylcellulose product NATROSOL® 250H NF grade are suitable materials.

The composition of the present invention may also contain one or more emulsifying agents which can be fatty acid esters such as polysorbate or polyols such as polyvinyl alcohol. The fatty acid esters preferably have a molecular weight greater than 1,000 daltons.

In some embodiments, the vasoactive vitamin is a vitamin B3 compound, such as niacin (nicotinic acid). It is preferred that when present the niacin be highly pure. In a further preferred embodiment, when niacin is present, vitamin B3 compounds other than niacin comprise less than 5%, and preferably less than 1.5% of the total vitamin B3 compounds in the composition.

Vitamin B3 compounds which are suitable in embodiments of the invention include the nicotinates. Specific nicotinates include benzyl nicotinate, inositol hexanicotinate, nicotinic acid, nicotinyl alcohol, xanthine nicotinate, methyl nicotinate, ethyl nicotinate, propyl nicotinate, isopropyl nicotinate, butyl nicotinate, isoamyl nicotinate, hexyl nicotinate, phenyl nicotinate, guaiacyl nicotinate, caffeine nicotinate, xanthinol nicotinate, nicametate citrate, nicotinamide, nicotinuric acid, nicotinyl hydroxamate, tocopheryl nicotinate, and mixtures thereof.

In one embodiment of the invention, the composition includes the vitamin B3 compound and an aqueous solution of p-hydroxybenzoic acid methyl ester, ethanol, methoxypolyethylene glycol, hyaluronic acid, hydroxyethylcellulose and carboxymethylcellulose and optionally one or more of propylene glycol, polyvinyl alcohol and polysorbate. In a further embodiment of the invention the composition includes, in addition to the vasoactive agent, the percent by weight: p-hydroxybenzoic acid methyl ester from about 0.1% to about 1%; ethanol from about 6% to about 10%; methoxypolyethylene glycol from about 5% to about 20%; propylene glycol from about 2% to about 5%; hyaluronic acid from about 0.05% to about 20%; hydroxyethylcellulose from about 0.1% to about 2%; carboxymethylcellulose from about 0.1% to about 2%; and naturally occurring or synthetic vitamin B3 compound from about 0.05% to about 15%.

In a further embodiment of the invention the aqueous solution includes p-hydroxybenzoic acid methyl ester, ethanol, methoxypolyethylene glycol, hydroxyethylcellulose, hyaluronic acid, carboxymethylcellulose and propylene glycol. In a still further embodiment of the invention from about 0.1% to about 1% p-hydroxybenzoic acid methyl ester; from about 6% to about 10% ethanol; from about 5% to about 10% methoxypolyethylene glycol; from about 2% to about 4% propylene glycol; from about 0.05% to about 20% hyaluronic acid; from about 0.1% to about 2% hydroxyethylcellulose; from about 0.1% to about 2% carboxymethylcellulose; and from about 0.05% to about 15% naturally occurring or synthetic vitamin B3 compound all in percent by weight are present.

In a further embodiment of the invention the composition includes an aqueous solution of p-hydroxybenzoic acid methyl ester, ethanol, methoxypolyethylene glycol, hyaluronic acid, hydroxyethylcellulose, carboxymethylcellulose, polyvinyl alcohol and polysorbate. In a preferred embodiment of the invention the composition comprises, in percent by weight: p-hydroxybenzoic acid methyl ester from about 0.1% to about 1%; ethanol from about 6% to about 10%; methoxypolyethylene glycol from about 10% to about 25%; polyvinyl alcohol from about 0.5% to about 3%; polysorbate from about 2% to about 8%; hyaluronic acid from about 0.5% to about 20%; hydroxyethylcellulose from about 0.1% to about 2%; carboxymethylcellulose from about 0.1% to about 2%; and naturally occurring or synthetic vasoactive vitamin from about 0.05% to about 15%.

The composition components may be combined in accordance with the following protocol. Table 1 describes the components in a composition of the invention including ranges for the concentrations of the components in the composition.

TABLE 1

| Compound | W/W % |
|---|---|
| Methyl Paraben | 0.1%-0.5% |
| Absolute Ethanol | 6%-10% |
| Methoxypolyethylene Glycol | 5%-20% |
| Hyaluronic Acid | 0.5%-25% |
| Hydroxyethylcellulose HW | 0.1%-2% |
| Carboxymethylcellulose HW | 0.1%-2% |
| Propylene Glycol (optional) | 2.5%-5.0% |
| Polyvinyl Alcohol (optional) | 0.1%-2% |
| Polysorbate 80 (optional) | 2%-5% |
| Vasoactive vitamins | 0.50%-15% |
| Purified Water USP | balance |

USP grade water is heated to about 90° C. and the heat is turned off. Methyl paraben (free base) is added to the heated water for 2 hours while stirring at about 450 rpm. Hydroxyethylcellulose and carboxymethylcellulose are mixed together in equal proportions by weight. When the temperature of the aqueous composition is about 70-80° C., an equal proportional mixture of hydroxyethylcellulose and carboxymethylcellulose is added while stirring at 450 rpm for about 30-35 minutes. When the temperature of the aqueous composition cools to about 55-60° C., methoxypolyethylene glycol is added while stirring at about 450 rpm for 6 hours. In a separate stirrer, the niacin is dissolved in a suitable solvent or is added neat to the aqueous composition at about 300 rpm for 1 hour. If there is a niacin solution, it is added to the aqueous composition while stirring at about 300 rpm for 1 hour. Propylene glycol is added to the aqueous composition while stirring at about 300 rpm for 1 hour. After the 1 hour, the stirring speed is reduced to about 90 rpm and the stirring of the aqueous composition continues overnight. The completed composition is packaged in suitable containers, such as polypropylene tubes. To maintain stability of the niacin during long term storage, the composition is stored at −15° C. to −20° C. On thawing, the composition is stable at room temperature for up to 30 days although the composition can be refrozen if storage for a longer period of time is desired.

For the stirring steps of the above procedure, stirrers that are capable of maintaining a constant stirring speed even when the viscosity of the solution is increased may be used. Such stirrers are referred to as fixed torque stirrers. Suitable stirrers include model BDC303 produced by Caframo Limited of Wiarton, Canada.

The composition of the present invention is a viscous aqueous gel-like solution. The gel like nature of the composition is maintained when the composition is frozen and thawed. When used for the treatment of female sexual dysfunction the composition is preferably applied to the perineal area of the female host, in particular the vagina and clitoris. This allows the vasoactive vitamin to increase the blood flow to these tissues resulting in the increased production of lubrication and increased tactile sensitivity, thereby improving the sexual function of the host. In various embodiments of the present invention, a single dose of the composition is about 2-3 ml and should be applied shortly before sexual activity, preferably not more than an hour before sexual activity. The effect of the vasoactive lasts for about one hour. After the one hour period the composition may be reapplied if necessary.

In the case of the treatment of erectile dysfunction, the composition is preferably applied directly to the penis so that blood flow can be increased leading to an improved erection. In various embodiments, the dose is preferably about 2-3 ml and is applied shortly before sexual activity, such as within about ten minutes prior to sexual activity.

In addition to application of the composition directly to the genital area, the composition may be provided on the surface of devices which contact the genital area during sexual activity, such as sexual aids or toys or devices used for birth control or prevention of sexually transmitted diseases. For example, the composition may be supplied on the outside and/or the inside of a condom. When supplied on the inside of a condom, the composition can contact the penis after application of the condom. When provided on the outside of a condom, the composition can contact a woman's vagina and/or clitoral regions during intercourse. Similarly, the composition may be supplied on the outside and/or inside of a female condom. In such uses, the composition on the outside of the female condom contacts the woman's vagina and/or clitoral regions. During intercourse, the composition on the inside of the female condom contacts the man's penis.

Improvement in sexual function can be evaluated by the use of questionnaires or diaries where the patient records the quality of their sexual experiences. Data collection should include a pretreatment baseline period of 4-8 weeks. Specific endpoints of the sexual experience include satisfactory sexual intercourse, sexual intercourse resulting in orgasm, oral sex resulting in orgasm and partner-initiated or self masturbation resulting in orgasm. A statistically significant change in the frequency of successful and satisfactory sexual experiences over time provides a measure of the effectiveness of the treatment for sexual function. The determination of what is a successful and satisfactory sexual experience is made by the patient, not the patient's partner. Physical changes such as increased blood flow to the genitals, engorgement of the penis or clitoris or changes in vaginal lubrication can also be measured but should be considered as secondary supportive information and not a primary indication of the success or failure of the treatment.

Questionnaires which can be used to evaluate sexual function include the Female Sexual Distress Scale described by Derogatis et. al. (J. Sex Marital Ther. 28(4):317-30, 2002) and the Female Sexual Function Index described by Rosen et. al. (J. Sex Marital Ther. 26(2): 191-208, 2000). And for males the International Index of Erectile Function (IIEF) described by Rosen et. al. (Int. J. Impotence Res. 11:319-326, 1999) can be used.

1. A composition for topical delivery to a host comprising an aqueous formulation of one or more vasoactive vitamins, one or more of an alkyl hydroxybenzoate preservative, an alkanol solvent, an alkoxylated alcohol humectant, and a high molecular weight hydrophilic colloid viscosity increasing agent.

2. The composition of the previous paragraph further comprising one or more emulsifying agents.

3. The composition of paragraph 1 wherein: (a) the alkyl hydroxybenzoate preservative is methyl paraben which is from about 0.1% to about 0.5% by weight of the composition, (b) the alkanol solvent is ethanol which is from about 6% to about 10% by weight of the composition, (c) the alkoxylated alcohol humectant is methoxypolyethylene glycol which is from about 5% to about 25% by weight of the composition, (d) the high molecular weight hydrophilic colloid viscosity increasing agent is selected from one or more of the group consisting of carboxymethylcellulose and hydroxyethylcellulose and which is from about 0.1% to about 2% by weight of the composition.

4. The composition of paragraph 1 wherein: (a) the alkyl hydroxybenzoate preservative is methyl paraben which is from about 0.1% to about 0.5% by weight of the composition, (b) the alkanol solvent is ethanol which is from about 6% to about 10% by weight of the composition, (c) the alkoxylated alcohol humectant is methoxypolyethylene glycol which is from about 5% to about 10% by weight of the composition, (d) the high molecular weight hydrophilic colloid viscosity increasing agent is selected from one or more of the group consisting of carboxymethylcellulose and hydroxyethylcellulose and which is from about 0.1% to about 2% by weight of the composition.

5. The composition of paragraph 2 wherein: (a) the alkyl hydroxybenzoate preservative is methyl paraben which is from about 0.1% to about 0.5% by weight of the composition, (b) the alkanol solvent is ethanol which is from about 6% to about 10% by weight of the composition, (c) the alkoxylated alcohol humectant is methoxypolyethylene glycol which is from about 10% to about 25% by weight of the composition, (d) the high molecular weight hydrophilic colloid viscosity increasing agent is selected from one or more of the group consisting of carboxymethylcellulose and hydroxyethylcellulose and which is from about 0.1% to about 2% by weight of the composition, (e) the fatty acid ester emulsifying agent is polysorbate 80 which is from about 2% to about 8% by weight of the composition.

6. The composition of paragraph 1 wherein the composition is an aqueous gel material.

7. The composition of paragraph 1 wherein the vasoactive vitamin is a naturally occurring or synthetic vitamin B3 compound.

8. The composition of paragraph 7 wherein the naturally occurring or synthetic vitamin B3 compound is niacin.

9. The composition of paragraphs 7 or 8 wherein the naturally occurring or synthetic vitamin B3 compound or niacin is from about 0.05% to about 15% percent by weight.

10. The composition of paragraph 9 wherein vitamin B3 compounds other than the niacin comprise less than about 1.5% by weight of the total vitamin B3 compounds.

11. A method for treating sexual dysfunction in a person comprising applying to the genitalia an aqueous formulation comprising one or more of a vasoactive vitamin, a alkyl hydroxybenzoate preservative, a alkanol solvent, a alkoxylated alcohol emulsifying agent, and a high molecular weight hydrophilic colloid viscosity increasing agent to a region of the body.

12. The method of paragraph 11 wherein: (a) the alkyl hydroxybenzoate preservative is methyl paraben which is from about 0.1% to about 0.5% by weight of the composition, (b) the alkanol solvent is ethanol which is from about 6% to about 10% by weight of the composition, (c) the alkoxylated alcohol humectant is methoxypolyethylene glycol which is from about 5% to about 25% by weight of the composition, (d) the high molecular weight hydrophilic colloid viscosity increasing agent is selected from one or more of the group consisting of carboxymethylcellulose and hydroxyethylcellulose and which is from about 0.1% to about 2% by weight of the composition.

13. The method of paragraph 12 wherein the person is a female and the composition is applied to the vagina.

14. The method of paragraph 12 wherein the person is a female and the composition is applied to the clitoris.

15. The method of paragraph 12 wherein the person is a female and the composition is applied to the vagina and the clitoris.

16. The method of paragraph 12 wherein the person is a male and the composition is applied to the penis.

17. The method of paragraph 12 wherein the composition is applied no more than about ten minutes before sexual activity.

18. The method of paragraph 12 wherein the composition is an aqueous gel material.

19. The method of paragraph 11 wherein the composition is applied to a condom that comes in contact with the genitalia of the person.

20. A method of formulating a composition for topical drug delivery comprising; (a) heating water to about 90° C., (b) adding p-hydroxybenzoic acid methyl ester to the water and, while the water is allowed to cool, stirring at about 450 rpm, (c) while the water is about 75° C. to about 80° C. adding hydroxyethylcellulose and carboxymethylcellulose and continue stirring at about 450 rpm, (d) while the water is about 55° C. to about 60° C. adding methoxypolyethylene glycol and continue stirring at 450 rpm, (e) dissolving a naturally occurring or synthetic vasoactive vitamin in an alkanol and adding the resulting alkanol solution to the aqueous solution with stirring at about 300 rpm, and (f) adding propylene glycol to the aqueous solution and stirring at about 300 rpm and then reducing the stirring speed to about 90 rpm.

21. The method of paragraph 20 wherein the composition is formulated using a fixed torque mixer.

22. The method of paragraph 20 wherein the naturally occurring or synthetic vasoactive vitamin is a vitamin B3 compound.

23. The method of paragraph 20 further comprising after adding the p-hydroxybenzoic acid methyl ester to the water the solution is stirred for about 2 hours.

24. The method of paragraph 20 further comprising after adding the hydroxyethylcellulose and carboxymethylcellulose the solution is stirred for from about 25 to about 40 minutes.

25. The method of paragraph 20 further comprising after adding the methoxypolyethylene glycol the solution is stirred for about 6 hours.

26. The method of paragraph 20 further comprising after adding the naturally occurring or synthetic vasoactive vitamin dissolved in ethanol to the aqueous solution the solution is stirred for about one hour.

27. The method of paragraph 20 further comprising after adding the propylene glycol to the aqueous solution the solution is stirred at about 300 rpm for about one hour and then stirring at about 90 rpm for from about 12 hours to about 18 hours.

28. The method of paragraph 20 further comprising packaging the composition in a tube.
